(19) United States
(12) Patent Application Publication
(10) Pub. No.: US 2018/0228403 A1
(43) Pub. Date: Aug. 16, 2018

(54) WEARABLE APARATUS FOR MONITORING HEAD POSTURE, AND METHOD OF USING THE SAME

(71) Applicant: Conghua Li, Markham (CA)

(72) Inventor: Conghua LI, Markham (CA)

(21) Appl. No.: 15/431,638

(22) Filed: Feb. 13, 2017

(51) Int. Cl. A61B 5/11; A61B 5/00

(52) U.S. Cl. CPC A61B 5/1116; A61B 5/746; A61B 5/7405; A61B 2562/0219; A61B 5/0004; A61B 5/6822; A61B 5/6823; A61B 5/7455

(57) ABSTRACT

A posture monitoring apparatus, comprising: at least one processor; and at least one sensor configured to sense position and posture of a wearer of the device; wherein the at least one processor receives and processes input from the at least one sensor, and where necessary, indicates the wearer's posture needs correction, and operates an alarm module to signal the wearer to correct the wearer's posture; wherein the apparatus is worn above the shoulders of the wearer.

### CROSS-REFERENCE TO RELATED APPLICATIONS

This application claims the benefit of the following provisional application, each of which is hereby incorporated by reference in its entirety: U.S. Pat. App. No. 62/294,544 filed on 12 Feb. 2016, U.S. Pat. App. No. 62/310,919 filed on 21 Mar. 2016, and Pat. App. No. 62/355,475 file on 28 Jun. 2016, all provisional applications entitled “WEARBALE APARATUS FOR IMPROVING NECK POSTURE AND METHOD OF USING THE SAME”.

### FIELD OF THE INVENTION

The present invention relates to wearable assemblies and methods for preventing, correcting, reducing and treating a range of disorders related to head posture and the nervous system. More specifically, the invention relates to rehabilitative and preventative care by helping users correct and improve posture while recovering from injury or improve posture during everyday activities.

### BACKGROUND OF THE INVENTION

The following review of the prior art is intended to provide edifying examples of corrective assembly as it relates to forward head posture and related disorders. The mention of these examples does not constitute an admission that any of the following methods or devices constitute prior art applicable to the present invention. The discussion of the references states what their authors assert, and the applicant reserves the right to challenge the accuracy and pertinence of any of the documents cited herein.

Posture disorders comprise a range of osteopathologies, neuropathies, and other ailments which can create chronic pain and disability. One such disorder is called Forward Head Posture (FHP). FHP, which is typically a subconscious and progressive condition, is characterized by the movement of a person's cranial alignment forward and/or downwards such that abnormal strain is placed upon the muscles, ligaments, fascia and bones of the upper body, especially around the neck and shoulders. This condition can become so severe that it impacts vital bodily functions and actually increases mortality rates among affected persons. The forward head carriage that is characteristic of FHP can result not only from genetic and/or age-related degeneration of the affected tissues, but also from repetitive poor head and neck posture. Its increasing prevalence in society is a result of the increasingly widespread, repetitive adoption of forward head postures among today's technology users, such as when a person reads at a desk, peers into a handheld device, or works at a computer terminal for extended periods of time on a daily basis.

Most attempts to correct posture are directed toward the spine, shoulders and pelvis. “Head position takes precedence over all others. The body follows the head. Therefore, the entire body is best aligned by first restoring proper functional alignment to the head.” Cailliet R, Gross L, Rejuvenation Strategy. New York, Doubleday and Co. 1987. “The extra pressure imposed on the neck from poor posture flattens the normal cervical curve resulting in abnormal strain on muscles, ligaments, fascia and bones” (American Journal of Pain Management, January 2008, 4:36-39). Persistent, forward-head posture increases compressive loads upon the upper thoracic vertebra, and is also associated with the development of Upper Thoracic Hump, which can devolve into Dowager Hump when the vertebra develop compression fractures (anterior wedging). FHP can lead to long term complications such as osteoarthritis. This disease promotes accelerated-aging of intervertebral joints resulting in degenerative joint disease. Posture impacts and modulates all bodily functions from breathing to hormonal production. Back pain, neck pain, headache, mood, blood pressure, pulse and lung capacity are among the many conditions influenced by faulty posture.

Current therapies for treating FHP and other posture disorders focus on corrective surgery, pain management, braces that mechanically force correct posture, exercises that focus on strengthening the neck muscles, physiotherapy, and chiropractic rehabilitation. These measures are costly and crude, and they are not effective in dealing with the subconscious nature of postural disorders. In light of the above, there exists a need for a method and assembly that both prevent and treat subconscious posture disorders like FHP in real time and in an economical, simplified, convenient, mobile, and effective manner.

A review of the prior art reveals a myriad of systems that detect and record postures. U.S. Pat. No. 9,196,175B2 granted to Walsh et al. disclosed a sensor pad that attaches to chairs and provides postural feedback to a user. U.S. Pat. No. 6,669,286 granted to Lusim disclosed a backrest with sensors that alarm users to poor posture. U.S. Pat. No. 6,673,027 granted to Fischer disclosed a hinge affixed to a user's sternum to record positions and alert a user to poor posture. U.S. Pat. No. 7,029,031 granted to Moisel et al. disclosed a sensor system that detected the position of a car passenger to facilitate safer air bag deployment in vehicles. U.S. Pat. No. 7,161,490 granted to Huiban disclosed a chair having back and arm rests with sensors that alarm users to poor posture. U.S. Pat. No. 7,471,290 to Wang, et al. disclosed posture sensors affixed to the torso but not the neck. U.S. Patent No. 20080049020 granted to Gusler, et al. disclosed a system to detect body position and adjust a computer monitor. U.S. Patent No. 20090058661 granted to Gleckler, et al. disclosed a system of pressure sensors in a chair to detect body positions.

Commercial products currently on the market are beginning to address posture issues and is referred to as ‘posture-correcting tech.’ The Up T-Shirt™ uses elastic bands imbedded into a t-shirt that pulls a user's shoulders when poor postures are performed. The Lumo Lift™ offers sensors that clip onto a user's clothing at the chest and records body positions and transmits them to mobile devices. The Prana™ offers a sensor on a waist clip that records posture and transmits it to mobile devices. The Arki™ offers a sensor bracelet that suggests posture corrections to a user while sitting. The Darma™ offers a seat cushion with sensors that record posture.

While inventions and products use sensors to detect body positions to improve postures they fail in the use of proximity sensors that are used with a user's neck area to measure movements of neck and/or head in the sagittal plane. Further, they fail to use accelerometers or gyroscopes that are associated around the ear area to measure the movements of the head and/or neck in the sagittal plane.

The present invention is an improvement from those other inventions and appliances because they are not mobile, not wearable, nor capable of monitoring user's neck or head posture while standing or walking, or in otherwise upright and mobile conditions, and are not capable of real-time monitoring user's neck or head postures.

### SUMMARY OF THE INVENTION

The present invention satisfies the aforementioned needs not found in the prior art and provides wearable therapeutic assemblies or FHP apparatus for real-time monitoring of head and neck postures—specifically targeting angles and distances in the sagittal plane. The present invention incorporates the use of proximity sensors, and/or accelerometers, and/or gyroscopes in the accomplishment of real-time monitoring. When forward shifts and/or inclines that deviate from pre-set ranges are detected over pre-determined threshold length of time, a user or a third party is alerted. The alert is generated by the assemblies themselves or by remote, electronic devices receiving transmissions from the FHP apparatus. The alerts raise a user's awareness of posture and may also be followed up with reminders, encouragement, obstructions of functions of electronic devices such as smartphones, computers and the like, and/or instructions on how a user can correct and improve their posture. As a result, users may prevent, reduce, or treat a range of disorders related to poor, upper-body posture—particularly FHP. Said FHP apparatus also tracks and analyzes the patterns of a user's neck and/or head posture over a pre-set period of time.

In one aspect, the present invention provides a posture and gait monitoring device, including: a processor; at least one sensor configured to sense position and posture of a wearer of the device; wherein the processor receives input from the at least one sensor indicating the wearer's posture needs correction and indicates that the wearer's posture or gait needs attention; wherein the device is worn above the shoulders of the wearer.

In an embodiment, the device includes an alarm module.

In an embodiment, the device includes a transmission module in communication with the processor and configured to indicate to an external device that the wearer's posture or gait needs attention.

In an embodiment, the transmission module instructs the external device to become unusable by the wearer until the wearer's posture is corrected.

In an embodiment, the alarm module comprises an auditory alarm mechanism.

In an embodiment, the alarm module comprises a vibratory alarm mechanism.

In an embodiment, the at least one processor processes posture and/or gait information received from the at least one sensor and compares the received posture and/or gait information with predetermined tolerances of space or time and/or predetermined patterns of linear and/or angular acceleration and/or velocity, and when the tolerances are exceeded and/or the patterns are matched, the processor controls an alarm module to indicate to the wearer and/or another party that attention to neck posture or gait is needed.

In an embodiment, the at least one sensor comprises selections from the group consisting of proximity sensors, touch sensors, accelerometers, and angular velocity sensors or gyroscopes.

In an embodiment, the at least one sensor measures angles and distances in the sagittal plane.

In an embodiment, the device includes a mounting mechanism.

In an embodiment, the device includes a collar attached to the mounting system, wherein the collar retains the processor, at least one sensor, and alarm module.

In an embodiment, the mounting mechanism comprises a vertically erect structure to which the at least one sensor is attached.

In an embodiment, the mounting mechanism includes a hinge that operates when the vertically erect structure makes contact with the wearer.

In an embodiment, the mounting mechanism comprises front pieces that extend toward the wearer's front and then downward until they are adjacent to the wearer's second rib.

In an embodiment, the device includes a chip attached to the at least one sensor, the chip configured to attach to a shirt collar worn by the wearer.

In an embodiment, the device includes a chip attached to the wearer's neck.

In an embodiment, the device includes a chip attached to the wearer's ear.

In an embodiment, the device includes a chip aligned with the at least one sensor, wherein the at least one sensor is configured to measure and monitor the distance between the chip and a target spot on the back of the wearer's neck using proximity sensors, wherein the chip is substantially vertical, and has a viewing/sensing beam that is substantially horizontal, wherein the target surface spot is located substantially on a gravity line of the wearer's neck and at the horizontal level of the at least one sensors inside the chip when wearer's neck tilts forward.

In an embodiment, the device includes a chip aligned with the at least one sensor, wherein the at least one sensor is tilted downward relative to the body of the chip from a horizontal position to a tilted position with a tilt angle approximating a medium tilt angle at which the wearer's neck tilts forward.

In another aspect, the present invention provides a method of correcting a person's neck posture, including the steps of: monitoring a person's neck posture by at least one sensor worn by a person; determining that the person's neck posture needs attention; and performing at least one of: drawing attention to the person's neck posture; and making an external device unusable until the person's neck posture is corrected.

In yet another aspect, the present invention provides a method of monitoring a person's gait, including the steps of: monitoring a person's gait by at least one sensor worn by a person; determining that the person's gait needs attention; and performing at least one of: drawing attention to the person's gait; and making an external device unusable by the person.

Many additional features and advantages of the present invention will become apparent from reading the following detailed description, when considered in conjunction with the accompanying drawings.

In theory, the wearer or user of the present invention can be any animal having both a neck and the cognitive ability to compensate for a sensory alert by adjusting its posture. The use of the present invention, for example, could be used to correct posture or behavior problems in mammals which are prone to such disorders, or it may be used to reproduce posture related disorders in mammals for experimental purposes, such as to create animal models of human posture disorders. Such animal models can lead to further therapies and treatments for FHP and related ailments in humans which cause great expense and suffering in societies throughout the world.

### Definitions of Terms

The term “body posture” or simply “posture” encompasses any condition or activity involving neuromuscular coordination, muscle tension (caused by holding a part of the body in a certain position), a sense of equilibrium or balance, the function of joints, biological mechanisms related to the movable parts of the body, and the like. Body posture is intended to be construed broadly. “Disorders related to body posture” is likewise intended to be construed broadly, encompassing disorders currently known as well as those not yet discovered.

The term “sensor” is used generally to refer to a sensing means to measure, detect, and/or monitor user's neck and/or head position, to measure, detect and/or monitor how far the position, location, and/or posture of user's neck and/or head is away from desired reference, to measure, detect, or monitor the period in which user's neck and/or head is in a certain position, including desired or undesired positions, and/or to detect and/or monitor user's state of being such as sitting, standing, walking, running, riding in a vehicle or flying in an aircraft. As referred to herein, sensor may include signal transmitters and receivers, and its corresponding signal processors. The term sensor may refer to both a singular and multiple sensors. The term sensor may refer to such sensor as, but not limited to, proximity sensors, distance sensors, time of flight sensors, accelerometers, angular velocity sensors, gyro sensors, tension sensors, pressure sensors, and/or contact sensors, and is intended to be construed broadly.

The term “vertically-erect structure” is used generally to refer to an upright erecting structure that supports, holds, carries, encases, and/or is associated with one or more sensors for proper positioning of the sensor to properly measure, detect and/or monitor the position of user's neck and/or head, and/or to measure the periods in which user's neck and/or head is in certain position in the sagittal plane. The vertically-erect structure is ideally flexible along its longitudinal direction to better accommodate user's backward neck and/or head movement, ergonomically shaped to conform to user's neck lordosis to allow easy wear and use in daily activities, extendable to allow said sensors to be properly aligned and/or associated with right part or parts of body to best detect neck and/or head location, and adjustable via a pivot mechanism to allow said vertically-erect structure to pivot forward and backward, to better detect neck and/or head position of users with various degrees of FHP, and to allow gradual correction or treatment of user's existing FHP by setting improving or higher levels of target reference that is closer and closer to an ideal good neck and/or head posture.

The term “situational appropriate” is used generally to describe the necessary fact that allowed or predetermined spatial and/or time tolerances need to vary depending on user's state of being such as sitting, standing, walking, running, driving and/or flying.

Parameters have been “therapeutically determined” whenever the geometric, spatial, time and/or energetic parameters required of an assembly of present invention have been determined by a physician, by the application of sound health care advice, by a desire for behavioral modification related to posture control, or by factors based upon a person's biology and/or need for therapy, health, wellness, comfort, and/or safety protection.

In order for the present invention to have reliable behavior, modification effects, therapeutic, and/or safety value, the sensors are placed along user's gravity line in the frontal plane, and behind or before user's neck, mounting mechanism one or both side of user's head substantially around the area of user's ear(s), in and/or on a substantially secure and/or stable supporting or mounting mechanism. A large-degree of unpredictability or random drift in the position of the sensors would be counterproductive. A simple means for achieving the stable emplacement of a sensor behind or before user's neck (or on one side or both sides of user's head), comprises one or more optional sensor holders and such mounting mechanism as, but not limited to, an upright erect structure, a set of straps to strap around user's trunk or shoulders, an arch like structure resting on user's shoulders, a shirt and/or a shirt collar, a set of medical Velcro pieces, and/or ear-bud-like or ear-ring-like structure. Any of these examples, as well as any method or assembly generally, which successfully position the sensors in a substantially secure and/or stable position behind or in front of user's neck, and/or on one side or both sides of user's head can position the sensors “upon” the wearer or user. “Substantially stable position”, as used herein, is not exclusive of also having adjustability. An adjustable mounting mechanism or an adjustable sensor holder such as, but not limited to, the vertically-erect structure can be adjusted to adopt a plurality of “substantially stable” positions.

The foregoing summary has outlined some features consistent with the present invention in order that the following detailed description thereof may be better understood, and in order that the present contribution to the art may be better appreciated. The present invention is not limited in its application, details, or components merely to those set forth in the following description and illustrations. The present invention resides not merely in any one of the features set forth in this specification, but also in the particular combination of all of the features and improvements claimed. Methods and devices consistent with the present invention are capable of other embodiments. Also, the phraseology and terminology employed herein are for the purpose of description and should not be regarded as limiting unless explicitly stated as such.

### BRIEF DESCRIPTION OF THE DRAWINGS

FIG. 1 is a perspective view of a first embodiment of the apparatus of the present invention where the sensing means is integrated on a mounting mechanism.

FIG. 2 is a front view of said first embodiment.

FIG. 3 is a side view of said first embodiment.

FIG. 4 is a side view of said embodiment mounted on a user wherein user's head is tilted forward.

FIG. 5 is a front view of said first embodiment mounted on a user.

FIG. 6 is an illustrative view of said first embodiment with peripheral elements of an embodiment of overall eco system.

FIG. 7 is a perspective view of another embodiment of the apparatus of the present invention where the rear part of the mounting mechanism has a lowered clearance void.

FIG. 8 is a perspective view of another embodiment similar to embodiment in FIG. 7, wherein front pieces 25 and/or 26 are equipped with horizontal extension/extensions 72.

FIG. 9 is a perspective view of another embodiment of the apparatus of the present invention.

FIG. 10 is a rear view of yet another embodiment of the apparatus of the present invention mounted on a user.

FIG. 11 is a side view of said embodiment in FIG. 10.

FIG. 12 is a rear view of yet another embodiment of the apparatus of the present invention mounted on an user.

FIG. 13 is a side view of said embodiment in FIG. 12.

FIG. 14 is a rear view of another embodiment of the apparatus of the present invention mounted on an user.

FIG. 15 is a side view of said embodiment in FIG. 14 mounted on user.

FIG. 16 is a side view of said embodiment in FIG. 14.

FIG. 17 is a rear view of another embodiment of the apparatus of the present invention mounted on an user.

FIG. 18 is a side view of another embodiment of mounted on user.

FIG. 19 is a front view of another embodiment

FIG. 20 is a perspective view of another embodiment of the apparatus of the present invention.

FIG. 21 is a perspective view of yet another embodiment of the apparatus of the present invention.

FIG. 22 is a perspective internal assembly view of said embodiment of the apparatus of the present invention in FIG. 20.

FIG. 23 is a schematic block diagram of an embodiment in FIG. 1, or FIG. 20

FIG. 24 is a schematic block diagram of another embodiment of the apparatus of present invention in FIG. 1 or FIG. 20

FIG. 25 is a schematic block diagram of another embodiment of the apparatus of present invention in FIG. 37, or FIG. 39

FIG. 26 is a schematic block diagram of another embodiment of the apparatus of present invention in FIG. 37, or FIG. 39

FIG. 27 is a schematic block diagram of another embodiment of the apparatus of present invention in FIG. 37 or FIG. 39

FIG. 28 is a front view of an embodiment of the apparatus of the present invention in FIG. 20, mounted on a collar.

FIG. 29 is a side view of an embodiment of the apparatus of the present invention in FIG. 20, mounted on a collar.

FIG. 30 is a side view of an embodiment of the apparatus of the present invention in FIG. 20, mounted on a collar.

FIG. 31 is a rear view of a collar with a special hole at the back.

FIG. 32 is a side view of a collar with a special hole at the back.

FIG. 33 is a side view of an embodiment of the apparatus of the present invention in FIG. 20, mounted on a collar and applied to a user.

FIG. 34 is a side view of an embodiment of the apparatus of the present invention in FIG. 20, mounted on a collar and applied to a user, when user's head protrudes forward.

FIG. 35 is a side view of a simplified simulation of proximity sensing arrangement of an embodiment of the apparatus of the present invention according to FIG. 20, FIG. 29 and FIG. 34.

FIG. 36 is a side view of a simplified illustrative simulation of proximity sensing arrangement of another embodiment of the apparatus of the present invention according to FIG. 20, FIG. 29 and FIG. 34.

FIG. 37 is a side view of an embodiment of the apparatus of the present invention according to FIG. 20, FIG. 25, FIG. 26, or FIG. 27.

FIG. 38 is a side view of an embodiment in FIG. 37, when user's head tilts forward.

FIG. 39 is a side view of another embodiment of the apparatus of the present invention similar to FIG. 37.

FIG. 40 is a perspective view of another embodiment of the apparatus of the present invention.

FIG. 41 is a side view of another embodiment of the apparatus of the present invention shown in FIG. 40, applied on a user.

FIG. 42 is a rear view of another embodiment of the apparatus of the present invention shown in FIG. 40, applied on a user.

### DETAILED DESCRIPTION OF THE DRAWINGS

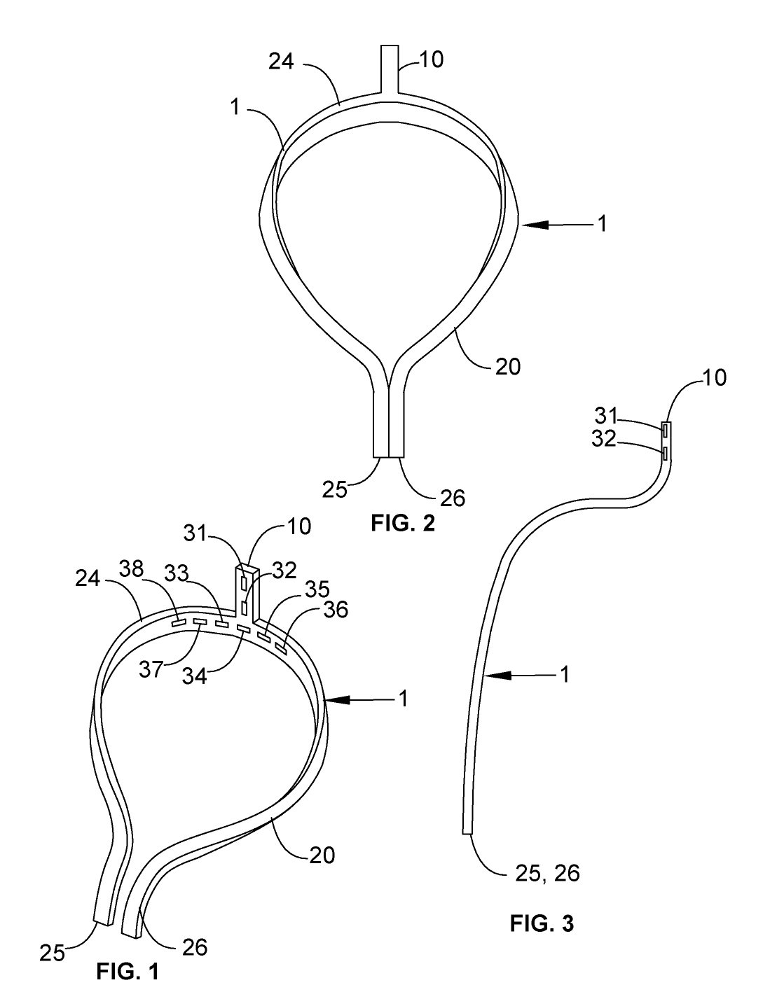

Directing attention generally to the figures, FIG. 1 is a perspective view of a first embodiment of the FHP apparatus 1 of the present invention, where the vertically erect structure 10 is adjustably associated with the mounting mechanism 20 in the center of the rear part of the mounting mechanism 20.

Vertically erect structure 10 can pivot forward and backward around its joint (not shown) with the mounting mechanism 20. The vertically erect structure 10 can also be adjusted by extension and contraction so that total height can be higher or lower (not shown). Vertically erect structure 10 may be made of rigid, semi-rigid or flexible material to allow backward bending to accommodate user's need to tilt his head backward occasionally, yet allow secure holding and positioning sensors associated with it.

Upper sensors 31 and lower sensors 32 are associated with, ideally held by, supported by, attached onto, and/or encased in vertically erect structure 10. Circuit board 33, buzzer/vibrator 34, communication module 35, power source unit 36, logic and operational control unit (microprocessor) 37, and/or power management integrated circuit (PMIC) 38, and combination thereof, are associated with, ideally held by, supported by, attached onto, and/or encased in mounting mechanism 20, or in vertically erect structure in space permits. Vertically erect structure 10 may be merged with mounting mechanism 20.

Sensor 31, sensor 32, circuit board 33, communication module 35, buzzer/vibrator 34, power source 36, logic and operation controller 37, and power management integrated circuit 38 are all to be associated electronically for desired functions.

Sensors 31 and 32 may be selected from sensors including proximity sensors, distance sensors, or contact or touch sensors.

In a preferred embodiment, sensors 31 or 32 are proximity sensors. Proximity sensors can be selected from the group consisting of, without being limited to, capacitive sensors, infrared sensors, time of flight sensors, ultrasound sensors, radar, inductive sensors, or laser sensors.

Touch or contact sensor may comprise such sensors as, but not limited to, capacitance sensor, resistance sensor, or Piezo touch sensor.

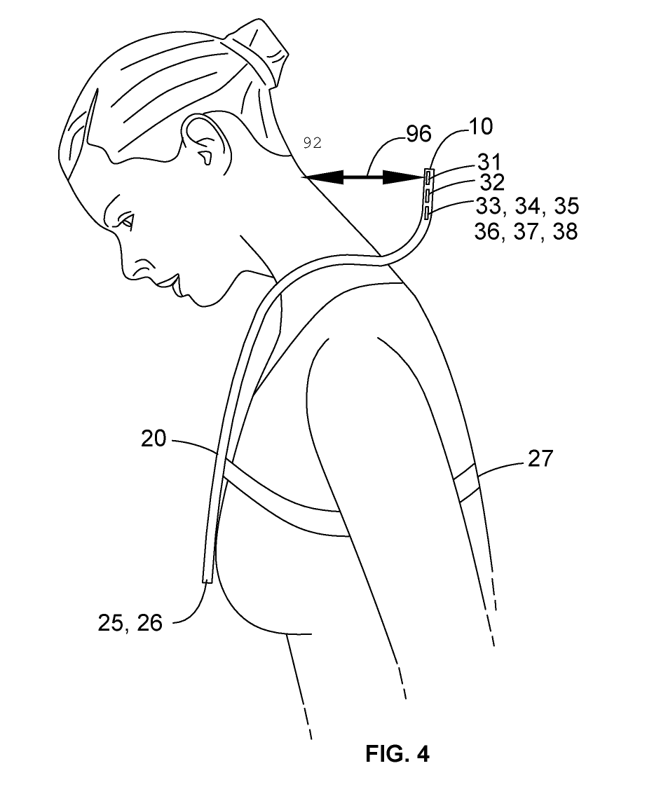

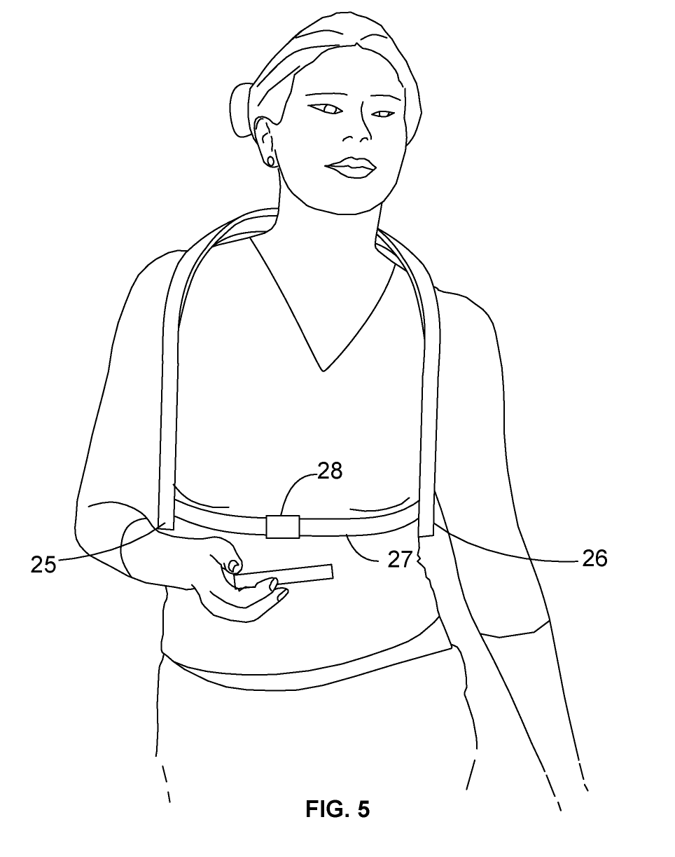

Mounting mechanism 20 has such shapes as, but not limited to, a loop, a semi-loop, or an arch, with a rear part 24, and front or side pieces 25 and 26. Mounting mechanism 20 is ergonomically designed and shaped to allow desired conformation to user's neck and/or body, desired grip to user's neck and/or body, secure holding of, secure support of, and/or easy operation of vertically erect structure 10 and other components such as, but not limited to, circuit board 33, communication module 35, buzzer/vibrator 34, and/or power supply 36. To improve said grip, said mounting mechanism 20 may be equipped with one or more adjustable straps 27 (described in FIG. 4 and FIG. 5) to loop around a part of user's body. Said one or more looping adjustable straps 27 may be equipped with one or more fastening means 28 (described in FIG. 4 and FIG. 5) for easy and secure operation. Said mounting mechanism 20 may be formed of rigid, semi-rigid, and/or flexible materials, or a combination of rigid, semi rigid, and/or flexible materials. Its back part 24 and front pieces 25 and 26 may be equipped with adjustable means to improve their grip onto user's body, or to improve the positioning of vertically erect structure 10 at the back of user's neck. In another embodiment, said front pieces 25 and 26 may be made of soft materials such as, but not limited to, fabric, tape, or strap.

In another embodiment, said mounting mechanism 20 may be made in such materials as, but not limited to, woven fabrics, non-woven fabrics, meshes, netting, webbing, fibers, leather, plastic, rubber, metal, and paper, in such forms as, but not limited to, clothes, clothing, shirt, vest, collar, neck tie, neck lace, and scarf.

In another embodiment, sensors 31 and 32 may be associated with mounting mechanism 20 or vertically erect structure 10 in such ways as, but not limited to, printing, gluing, pressing, embossing, woven, clipping, stapling, fastening, and magnetic locking. Circuit board 33 is preferably a printed circuit board (PCB) to associate and/or integrate all electronic, power, transmission components of FHP apparatus 1.

Circuit board 33 includes one or more pieces and one or more layers of circuit boards, one or more printed antenna for data/signal transmission to external electronic devices, and all electronic components of FHP apparatus 1. Buzzer/vibrator 34 is controlled by logic and operation controller 37, and provides alert based on predetermined logics and algorithms, and is preferably a low power actuator for sending sensory alert while not consuming too much power.

To be discussed later, communication module 35 is used to transmit data/signals to and communicate with one or more external electronic devices. The communication module 35 is preferably a wireless module.

Power source 36 is preferably a rechargeable battery that desirably has a power supply of 90 mAH or more, for a sufficient period of operation. Power management integrated circuit (PMIC) 38 provides such necessary functions as, but not limited to, charger, voltage conversion, power saving operation, time clock, fuel gage, and/or any combination thereof.

Logic and operation controller 37 is a specialized microprocessor that controls and manages the operations of the sensors and other components of FHP apparatus 1 according to predetermined logic, algorithm, and/or parameters, for continued real time monitoring user's head posture during various daily activities.

FIG. 2 is a front view of said first embodiment of the FHP apparatus 1.

Vertically erect structure 10 and mounting mechanism 20 are designed according to minimalistic and aerodynamic principle, to achieve best possible conformation to user's body, and to achieve minimal irregular outward bulging and potential interference with clothing that user may wear on top of or underneath said apparatus 1. Said mounting mechanism 20 is substantially designed in a form of a tennis racket head. The front pieces 25 and 26 may be joined together at their lower portion with a fastening means (not shown). Ideally the front pieces extend toward front and then downward till the level of second rib, and then, if necessary, turn towards the center line of the body, and after joining together, turn downward till their bottom ends reach the level of third or fourth rib, for best grip to user's body. In this embodiment the mounting mechanism 20 and vertically erect structure 10 are designed and associated in a symmetrical manner with vertically erect structure 10 located at the center of the rear part 24 of the mounting mechanism. Depending on user's needs, they may also be presented in an asymmetrical manner. For user's comfort, there may be a hypoallergenic padding layer (not shown) underneath said mounting mechanism 20 to allow soft contact between the mounting mechanism 20 and user's skin.

FIG. 3 is a side view of the FHP apparatus 1 of the first embodiment. Mounting mechanism 20 protrudes forward and bend downwards with its front pieces 25 and 26, while vertically erect structure 10 protrudes upwards. Said vertically erect structure 10 may be designed to follow the concave curve of user's back neck for improved ease and comfort of use, and for improved sensing and monitoring user's neck and/or head position, in which case vertically erect structure 10 may concave towards the curvature of user's neck lordosis, with its top end extending upwards to reach the deepest concave area of user's neck (around vertebrae C3 which is just underneath the hair line on the back of user's head in most users). Such alignment helps improve accuracy of measuring the distance between sensors 31 and the back of user's neck, because, given the same angle of forward tilt of user's neck, said distance increases from the base to vertebrae C3 of user's neck.

FIG. 4 is a side view of said embodiment shown in FIG. 3 mounted on a user wherein user's head is tilted forward. The location of sensor(s) 31 and a sensing beam 96 remains substantially the same as before user's head is tilted forward. The distance between the sensor(s) 31 and the back of user's neck 92 increases as user's head tilts forward. The more the user's head tilts forward, the greater said distance. By directly measuring the change of said distance, the forward tilting of user's neck, hence the forward protrusion of user's head and user's neck posture, can be monitored.

In comparison to the prior art, the present invention performs direct measurement and monitor of neck and head movement or location, and hence user's neck/head posture. This is far more effective than if sensors are placed elsewhere on a user's body in order to interpret user's neck or head movement/posture by measuring the movement or location of user's other body part/parts instead of user's neck or head.

Real-time monitoring of the movement of a user's neck 92 occurs, in one embodiment, through the use of a proximity sensor. In such embodiment, the proximity sensor emits a sensing beam, for example an infrared beam, which upon meeting the user's neck 92, bounces back to the sensor 31 or 32, a determination of movement or position of a user's neck 92 is based on the time it takes for a return signal to be read by the sensor 31 or 32. For example, if the time to receive the return signal is greater than a first return signal T₀, then the FHP apparatus 1 determines the user's neck 92 has moved in a forward direction. Other means for determining movement of a user's neck by the proximity sensor can be used, and are well-known to those with ordinary skill in the art. For example in case of capacitive sensors, if the signal is weaker than a previous signal received, a determination is made that a user's neck has moved forward. In use, the sensors 31 or 32, such as proximity sensors, monitor a user's neck movement by sending a signal at a predetermined rate, for example twice per second.

FIG. 5 is a front view of said first embodiment in FIG. 1 mounted on a user. Said mounting mechanism 20 loops around user's neck and rest of user's shoulders, with two front pieces 25 and 26 extending forwards and downwards from user's shoulders. To achieve more secure attachment to user's body, one or more straps 27 could be applied to the front pieces 25 and 26 to wrap around user's body. Strap 27 may be equipped with fastener 28 for ease of use and adjustment of tightness around user's body. Said straps 27 may be made of, but not limited to, fabric or webbing materials. Said fastener may be made of, but not limited to, quick release buckles or hook and loop pieces. FIG. 5 shows only one embodiment of strapping. Other strapping method could be applied by one skilled in the art to offer appropriate attachment of apparatus 1 on user's body.

FIG. 6 is an illustrative view of said first embodiment with peripheral elements of an embodiment of overall eco system for sensing, monitoring, alerting, instructing, documenting, tracking, analyzing, storing, reviewing, and otherwise processing user's neck and/or head posture data and/or information, to help user reduce, correct, or prevent FHP, and/or reduce and/or manage risks associated with lack of attention, awareness, bodily readiness, wherein the FHP apparatus 1 is within an overall ecosystem 100.

The overall eco system 100 may comprise one or more external electronic devices 51 and/or 52 such as, but not limited to, smartphones, smart wrest bends, smart finger rings, electronic readers, personal digital assistants (PDAs), personal music or multimedia devices, one or more external electronic devices 60 such as, but not limited to, computing servers, computing cloud, LANs, WANs, communication, alert or control devices such as, but not limited to, car, truck, bus, train, boat, airplane, home, office radios, broadcast systems, TV sets, seats, navigation devices, speed control system, engine control system, office work stations, or any combination or combinations thereof, and one or more electronic data processing, viewing, storing, devices 61 or 62 such as, but not limited to, smartphone, tablets, personal computers, lap top computers, desktop computers, tablet and/or computers. External electronic device such as 51 and/or 52 receives communication from communication module 35, process the received information, and trigger and/or present sensory alerts or posture correction recommendations or instructions depending on a therapeutically predetermined logic and parameters, record, store, analyze, and/or further transmit to other associated electronic devices such as, but not limited to, computing servers and/or cloud, user neck and/or head posture information. Said alert or posture correction recommendations may be auditory, visual, vibratory, interruption or shut down of operation of associated external electronic devices, and/or further sensory and/or non-sensory means. Associated external electronic devices 60 may receive user's postural data from one or more external devices such as 51 and 52, or may receive user's postural data directly from communication module 35, process the received information, and store, process, analyze, transform, translate, present, and/or further transmit said data, and may trigger or release certain predetermined or programmed communications, alerts, advice, or actions to remind, encourage or force user to improve their posture timely. The corresponding operations of the external electronic devices are controlled and/or managed by an app or software residing on the relevant external electronic devices. Said app or software need to be preinstalled on the relevant external electronic devices, and to be pre-paired with the FHP apparatus 1, before being able to work with said apparatus.

Communication between the FHP apparatus 1 and the ecosystem 100 can be accomplished through a variety of wired and wireless means, known in the art, with the wireless means selected from such group of protocols consisting of, but not limited to, Zigbee™, Insteon™, Zwave™, WIFI™, Bluetooth™, and BLE™ (Bluetooth Low Energy).

For example, if a user is driving a car, and tilts her head downwards for a period more than a predetermined length, her smartphone may send sensory alert, her smartphone operation may be interrupted by such means, but not limited to, large warnings across the screen that blocks user's view of screen, locking/freezing the operation of the smartphone, or shutting down the phone, the normal play of her car radio may be interrupted by loud and/or disturbing warnings or alarms, her navigation system may be subdued or be covered by large warnings, or be blacked out, she may not be able to start the engine if she was not driving, or, if she was driving, the speed of her car may be automatically reduced, or the cruise control may be released or blocked, or the vibrator in her car seat may begin to work in unpleasant manner, or the engine may even be forced to shut down, according to predetermined parameters, logics, and programs, for the safety of the user. As such, a user's biomechanical behaviors and/or conditions are to be linked with her vehicle, workstation, or equipment. The working of her car, her workstation and/or her equipment may be influenced or controlled by the user's biomechanical behaviours or conditions for the user's health and/or safety interests. Such link may be applied beyond the situations such as a user with her car, a pilot with her airplane, an office worker with her workstation, a senior and her home, a baby and her bed, but also be applied to such situation as a worker and his machines, a soldier and his equipment and/or his artificial limbs. Such monitoring may be well beyond the neck or head posture of the user, and may include such factors of the user as, but not limited to, user's postural stability, blood sugar level, blood alcohol level, heart rate, respiration, skin temperature, step frequency, walking patterns, duration of no motion, even eyelid motions. The result may be integrated real time monitoring of human behavior and conditions with improved protection of user health, safety, and/or productivity, and should put human behavior at the center of the “internet of things” which may be better described as “internet of behaviors and things”.

External electronic devices such as 61 or 62 may receive user's postural data from external electronic devices 60, from external electronic devices such as 51 and 52, and/or from communication module 35 directly, for viewing, storing, recording, analyzing, cataloging, classifying, archiving, visualizing, summarizing, and/or otherwise processing user's postural data, and/or for displaying, presenting, sending, and/or transmitting instructions and/or alerts, to encourage user improve his neck and/or head posture. All said elements are electronically associated according to one or more predefined logic, and whether or not wirelessly.

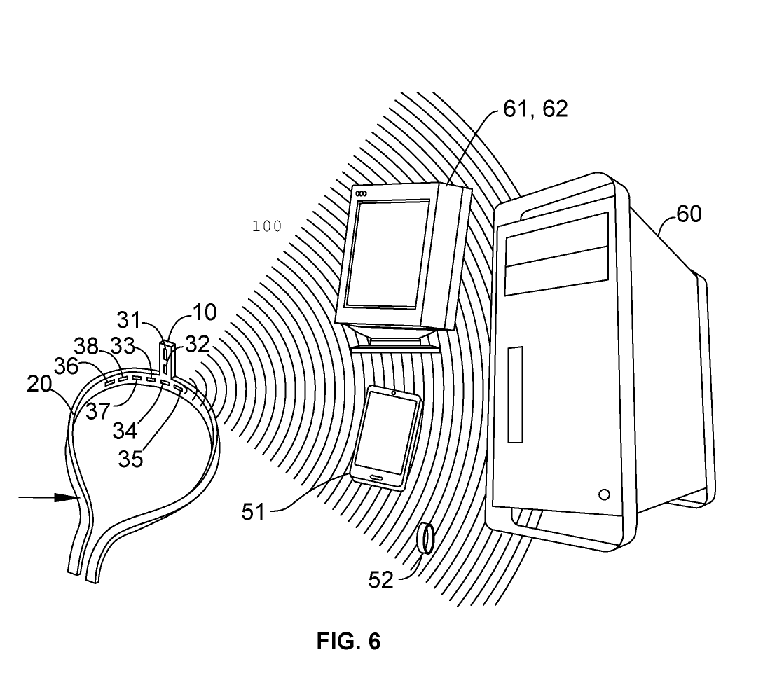

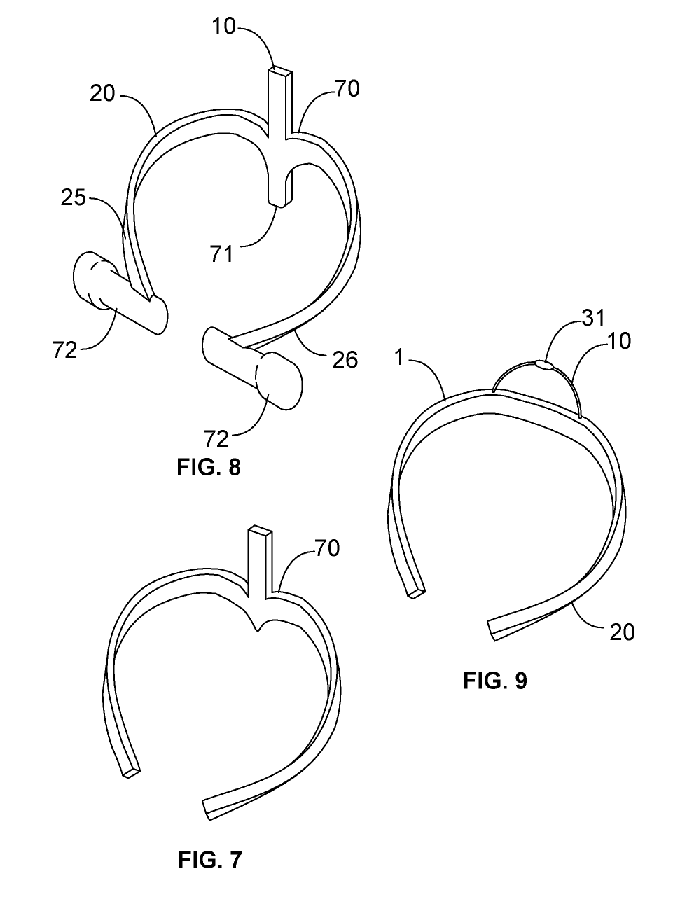

FIG. 7 is a perspective view of another embodiment of the FHP apparatus 1 of the present invention where the rear part of the mounting mechanism has a lowered clearance void 70. The lower end of void 70 which is the supporting base for vertically erect structure 10 may extend downwards to reach T2 or lower. Such void may help provide greater freedom to user's neck, reduce or eliminate pressure on user's neck, or improve the accuracy, reliability and/or stability of positioning sensors 31 or 32 behind user's neck to align the upper sensor with the deepest concave area of user's neck (around vertebrae C3 which is just underneath the hair line on the back of user's head in most users,), both vertically and in view of the distance between user's neck and sensor 31 and/or 32. Vertical alignment of top sensors 31 with vertebrae C3 may allow best sensing and monitoring user's neck and/or head position. In other embodiments, the vertical alignment of top sensor may be above or below vertebrae C3 along user's neck.

FIG. 8 is a perspective view of another embodiment of the FHP apparatus 1 of the present invention that is similar to embodiment in FIG. 7, but wherein the front parts are to press on user's frontal deltoid muscles around coracoid process, and lower part of the void structure reaches lower, to help user reduce forward rolling shoulders and encourage an open-chest posture. The front parts 25 and 26 may be equipped with horizontally extended parts 72 to allow gentle compression on user's front deltoid muscles and coracoid processes. The downward extension 71 reaches down along user's spine to the level of T4 or lower.

FIG. 9 is a perspective view of another embodiment of the FHP apparatus 1 of the present invention. Vertically erect structure 10 is in the form of an erected arch. Sensor 31 may be substantially centered on the top portion of the erected arch 10. Said arch 10 may be elastically flexible to move forward and backward of its user, or in other words, towards and/or away from user's neck. Other necessary electronics may be contained in mounting mechanism 20. Said arch 10 may be made of, but not limited to, flexible plastic, rubber or mental materials.

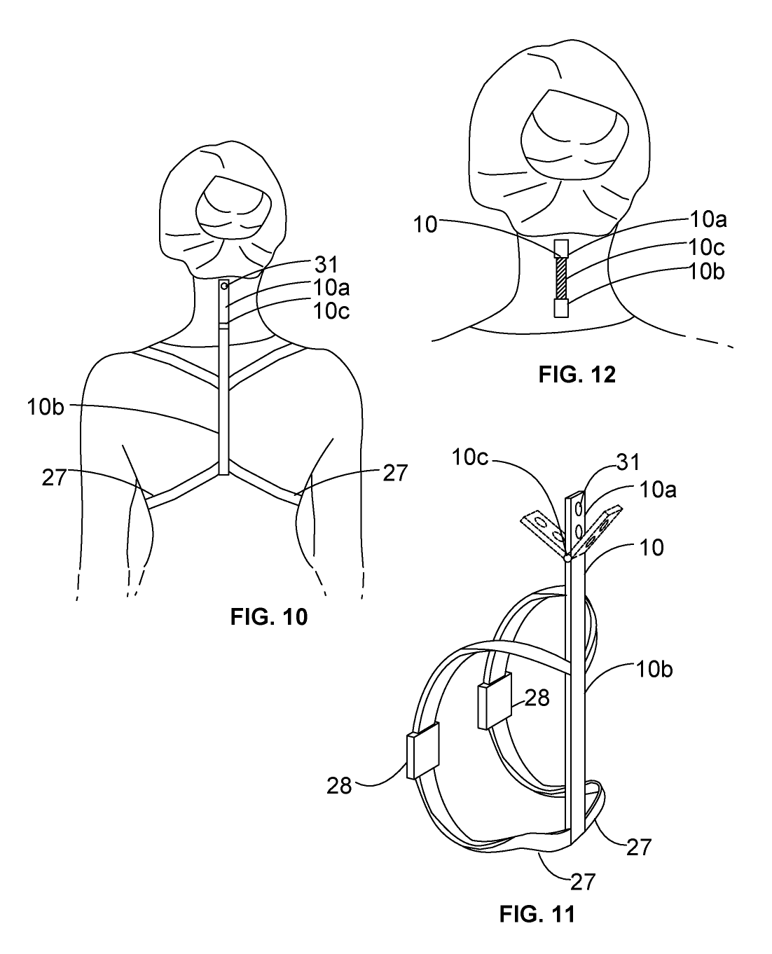

FIG. 10 is a rear view of another embodiment of the apparatus of the present invention mounted on an user. Vertically erect structure 10 is affixed to user's body through a pair of straps 27. In other words, mounting mechanism 20 is integrated or merged into said vertically erect structure 10, or has been simplified and reduced to comprise straps 27 only. Sensor 31 may be substantially located close to the top edge of the vertically erect structure 10. While in use, said top edge of vertically erect structure 10 is substantially aligned with the upper neck. Pivot section 10c is substantially aligned with the base of user's neck, around the level of vertebrae C7/T1 of user's spine.

FIG. 11 is a side view of said embodiment in FIG. 10. Vertically erect structure 10 may comprise an upper section 10a, a lower section 10b, and a pivot mechanism 10c. Upper section 10a can pivot around pivot mechanism 10c to improve fitting with user's neck and/or head. Sensor 31 or 32 may be associated with the top and/or middle portions of 10a. Both left and right straps 27 may be equipped with one or more fastening means 28 for quick mounting on and demounting from user's body. In this embodiment, mounting mechanism has been transformed into lower section 10b. Upper section 10a and/or lower section 10b may be made of, but not limited to, rigid and/or semi-rigid plastic, rubber, or metal materials. Pivot mechanism 10c is such a mechanism that is capable of easy adjusting and secure maintaining the erect angle of said upper section 10a with relation to lower section 10b.

FIG. 12 is a rear view of another embodiment of the apparatus of the present invention mounted on a user. Vertically erect structure 10 is substantially vertically and centered attached on the back of user's neck along user's spine, with its top end of upper section 10a aligned with, and securely and removably attached to the back of user's neck around vertebrae C3, i.e. the hair line at the back of user's head, and its bottom end of lower section 10b aligned with, and securely and removably attached to the back of user's neck around user's vertebrae T1 to T2. Vertically erect structure 10 comprises an upper section 10a, a lower section 10b and a tension sensing section 10c.

Tension section 10c comprises at least one tension and/or stretch sensor capable of sensing and/or measuring pulling tension and longitudinal stretch along vertically erect structure 10. Said stretch sensors may be for example, but not limited to, cable stretch sensor, fabric stretch sensor, knit stretch sensor, printed stretch sensor, paper stretch sensor, polymer film stretch sensor, conductive rubber stretch sensor. When user's head protrudes forward, the surface distance between vertebrae C3, i.e. the hair line at the back of user's head, and vertebrae T1/T2 on the back of user's neck increases, and causes vertically erect structure 10 to extend longitudinally. The extension of vertically erect structure 10 activates the tension and/or stretch sensors associated with tension sensing section 10c in response to stretching tension. Tension/stretch sensors send communication to logic and operational control unit or microprocessor 37 which, when user's head tilts forward beyond a predetermined spatial and/or time tolerance, may trigger, either immediately or after a predefined period, sensory alerts and/or instructions to user of undesirable neck and/or head posture from the apparatus 1, and/or from one or more external devices such as, but not limited to, a smartphone, a smart wrest bend, a smart finger ring, a tablet, a compute, or another portable or non-portable electronic device, and any combination thereof, to encourage user to improve his neck and/or head posture.

Such sensory alert and/or instructions may comprise for example, but not limited to, vocal, visual, motion, or other sensory alert. A delay in triggering sensory alerts and/or instructions may serve user's need of knowingly, periodically or randomly protruding forward the neck and/or head briefly, without being alerted of such forward protrusion.

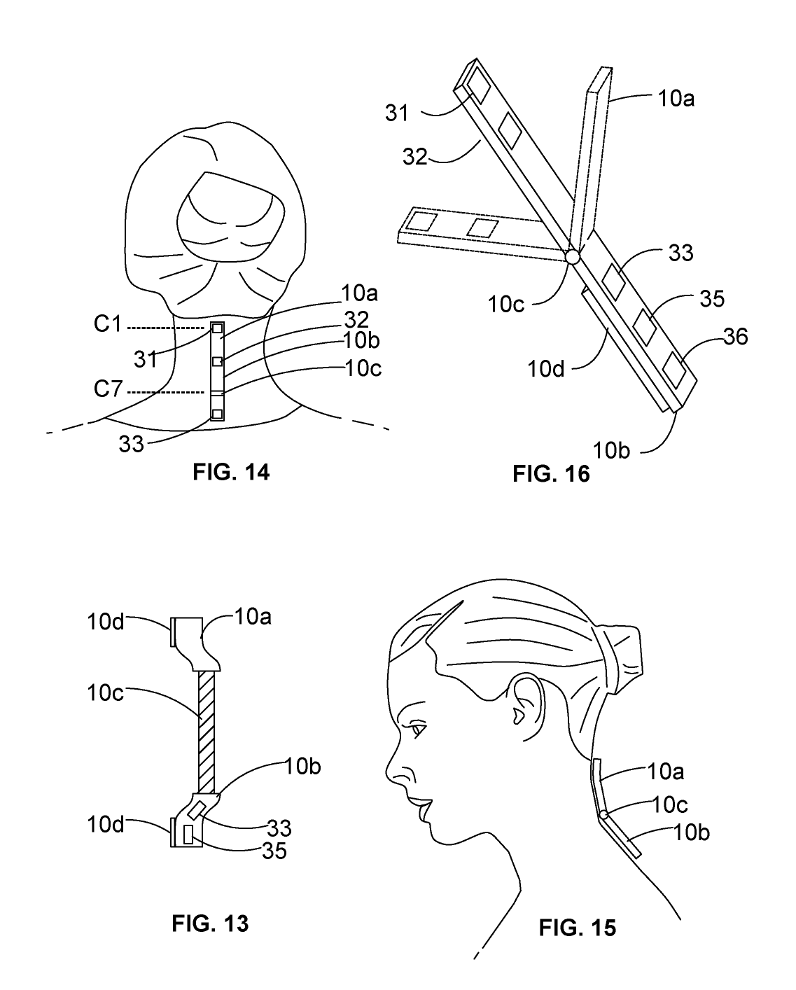

FIG. 13 is a side view of said embodiment in FIG. 12. Vertically erect structure 10 comprises an upper section 10a, a lower section 10b and a tension sensing section 10c. Tension section 10c comprises at least one tension and/or stretch sensor capable of sensing and/or measuring pulling tension and longitudinal stretch along vertically erect structure 10. Upper section 10a and lower section 10b have attaching means 10d and can be securely but removably attached on the back of user's neck. Said attaching means 10d may be, for example, but not limited to, one or more pairs of medical Velcro strips, adhesive tapes, or adhesive layer.

Tension sensing section 10c is capable of sensing and/or measuring stretching tension. It allows vertically erect structure 10 to extend longitudinally, when user's head protrudes forward beyond a set of therapeutically predefined tolerance parameters, and sends signals through a logic and operation controller 37 and/or a communication module to trigger sensory alert or instruction functions from one or more external electronic devices, or from an alert and/or instruction device integrated in itself, such as, but not limited to, Transcutaneous Electrical Nerve Stimulation (TENS) unit which intensity of stimulation may vary according to the degree of stretch of said apparatus, based on therapeutically predefined logics and parameters.

Such sensory device may also send sensory alerts and/or instructions to user to further help him improve his head and/or neck posture. Upper section 10a and/or lower section 10b may be made of, but not limited to, rigid or semi-rigid materials such as plastic, rubber or metal. In another embodiment, upper section 10a and/or lower section 10b may be made of, soft materials such as, but not limited to, polymer film, rubber strip, knitted materials, paper strip, or woven or nonwoven fabrics. In another embodiment, upper section 10a and/or lower section 10b may be made of stretchy materials such as, but not limited to, stretchy plastic film, stretchy rubber strip, stretchy knitted materials, stretchy paper strip, or stretchy woven or nonwoven fabrics. In another embodiment, upper section 10a, lower section 10b, tension section 10c, and/or any of their combinations may be merged together in one integral piece. In another embodiment, upper section 10a, lower section 10b, tension section 10c, and/or any of their combinations may be made in same stretch sensing design and/or materials in one integral piece. In another embodiment, upper section 10a, lower section 10b, tension section 10c, and/or any of their combinations may be made in same stretchy design and/or materials in one integral piece. In another embodiment, upper section 10a, lower section 10b, tension section 10c, and/or any of their combinations may be merged together and made of one or any combination of materials such as, but not limited to, knitted stretch sensor, or woven stretch sensing and/or measuring fabric sensor. In another embodiment, attaching means 10d may be applied to all of upper section 10a, lower section 10b, tension section 10c, or any of their combinations. Other embodiments comprise any combination of all and/or any of above discussed embodiments.

FIG. 14 is a rear view of another embodiment of the FHP apparatus 1 of the present invention mounted on a user. Vertically erect structure 10 comprises an upper section 10a, a lower section 10b and a pivot section 10c. Vertically erect structure 10 is attached to the back of user's neck via the lower section 10b. The top end of vertically erect structure 10 is aligned with user's deepest concave area of user's neck (around vertebrae C3 which is just underneath the hair line on the back of user's head in most users). The lower section 10b is attached to the back of user's neck via such means as, but not limited to, medical Velcro strips, adhesive tapes, adhesive layer, or straps. The pivot section 10c is aligned with user's vertebrae C7. Pivot section 10c is capable of easy adjusting and secure maintaining the erect angle of upper section 10a in relation to lower section 10b. Said adjustable angle range may be no less than 45 degree, with zero degree referring to the state where upper section 10a and lower section 10b are aligned in one straight line. Upper section 10a is adjusted to be substantially in touch with or in substantial close proximity to the back of user's neck, when user is in an upright and good posture with his neck and head.

When user's head protrudes forward, said touch is void, the distance between the top end of 10a and the back of user's neck increases, sensors 31 and/or 32 measure and send corresponding data/signals to logic and operation controller 37 which, when the forward protrusion of user's neck exceeds a predetermined spatial and/or time tolerance, may trigger, either immediately or after a predefined period, sensory alerts and/or instructions to user of undesirable neck and/or head posture from the FHP apparatus 1 and/or one or more external devices, as previously discussed, to encourage user to improve his neck and/or head posture. Such sensory alert and/or instructions may comprise for example, but not limited to, stimulation by a TENS unit, vibration, vocal, sound, visual, motion, or other sensory alert. A delay in triggering sensory alerts and/or instructions may serve user's need of knowingly, periodically or randomly protruding forward the neck and/or head briefly, without being alerted of such forward protrusion.

In another embodiment of present invention, sensors are flexion or bend sensors such as, but not limited to, conductive ink-based, fiber-optic, and/or conductive fabric/thread/polymer-based sensors, or any of their combinations. Said bend sensor it associated along the upper section 10a. Upper section 10a is made of flexible materials such as, but not limited to, flexible plastics, flexible rubber, and flexible metal. The surface of the top end of vertically erect structure 10 may be arranged to be reasonably securely yet removably attached to the back of user's neck around vertebrae C3 level. When user's head protrudes forward, said top end of vertically erect structure 10 may be pulled forward causing the said flexible upper section 10a to bend forward. Bend sensor may send communication to logic and operation controller 37 which may then trigger, either immediately or after a predefined period, sensory alerts and/or instructions to user of undesired neck and/or head posture from the apparatus 1 and/or one or more external devices, as previously discussed, to encourage user to improve his neck and/or head posture. Such sensory alert and/or instructions are as previously discussed. A delay in triggering sensory alerts and/or instructions may serve user's need of knowingly, periodically or randomly protruding forward the neck and/or head briefly, without being alerted of such forward protrusion.

FIG. 15 is a side view of said embodiment in FIG. 14 mounted on user. Upper section 10a concaves towards or with the lordosis on the back of user's neck. The top edge of upper section 10a may be substantially in touch with or close to the back of user's mid to upper neck when user is in a good and upright head/neck posture. Pivot section 10c may be substantially aligned with the base of user's neck around vertebrae C7/T1. Sensor and/or sensors may be associated with the upper section 10a and aligned with top edge of said upper section 10a.

FIG. 16 is a perspective view of said embodiment in FIG. 14. Vertically erect structure 10 comprises an upper section 10a, and lower section 10b and a pivot section 10c. Pivot section 10c allows easy adjustment of upper section 10a so that the upper end of 10a can be adjusted to be substantially in slight touch with or in substantial close proximity to the back of user's neck, when user is in a good upright posture with his neck and head. Pivot section 10c is capable of holding the position or angle of upper section 10a securely without undesired pivot movement either forward or backward. Lower section 10b can be affixed to the back of user's neck, as previously discussed. Sensor and/or sensors 31 and/or 32 may be integrated with the upper section 10a, sensors 31 substantially aligns with the top edge of said upper section 10a. Buzzer/vibrator 34, and power source or battery unit 36, communication module 35, circuit board 33, logic and operation controller 37, and power management integrated circuit (PMIC) 38 may be integrated with either upper section 10a or lower section 10b.

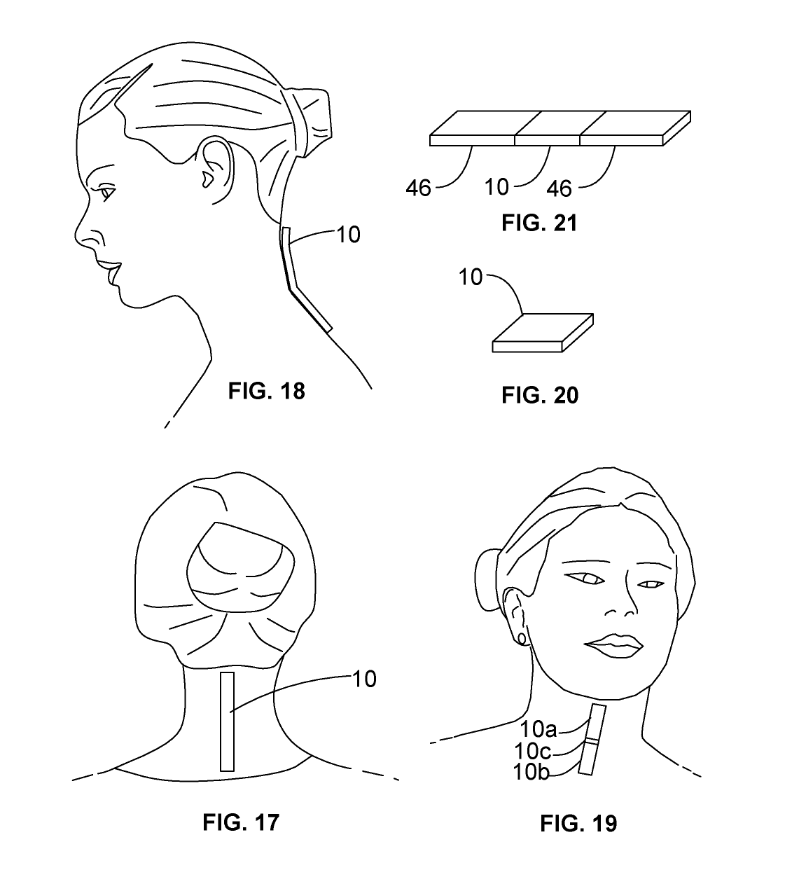

FIG. 17 is a rear view of another embodiment of the FHP apparatus 1 of the present invention mounted on a user. Said apparatus 1 is substantially a flexible and/or elastic tape which may or may not be integrated with sensor or sensors. When present, such sensor or sensors may be integrated with said tape through such method as, but not limited to, weaving, coating, laminating, or printing. Upper end of said apparatus is applied to the center line of user rear neck at the level of vertebrae C3. Lower end of said apparatus 1 extends downwards to T1 or lower. Said embodiment of apparatus 1 is applied on user's rear neck through such method as, but not limited to, glue and/or hook and loop strips. When user's head protrudes forward, said apparatus may be pulled and/or extended longitudinally. Such pull and/or extension may create myofascial sensation of stretching on the skin on the back of user's neck, which may alert user of inappropriate head or neck posture, and encourage user reduce the forward protrusion of his head/neck. When stretch sensor or sensors are present in said apparatus 1, said sensor or sensors may emit signals that indicate user's head protrudes forward and said apparatus 1 is extended longitudinally. Such signals may be sent to logic and operation controller 37 (not shown) that may trigger sensory alerts and/or instructions from within said FHP apparatus 1 itself, or one or more of external electronic device, as previously discussed. Said sensory alert may encourage user to reduce the forward protrusion of his head. In another embodiment, said FHP apparatus 1 may consist one or more pieces of tape applied on the back of user's neck simultaneously. Said tape-like apparatus 1 may be securely applied to the back of user's neck via adhesive. A set of tape pieces with varied widths and/or varied degrees of elasticity may be applied on the back of user's neck simultaneously. Said elastic tape like FHP apparatus 1 may be applied with certain predetermined tension by pre-stretch the tape before application on the back of user's neck. Such predetermined tension may help increase user's awareness of a good posture even when his head is not protruded forward. Ideally the width of each tape piece may be in the range from 10 mm-40 mm. As previously discussed, the FHP apparatus 1 may comprise accelerometers, and/or angular velocity sensors.

FIG. 18 is a side view of another embodiment of FHP apparatus 1 mounted on a user. Said sensor is applied to the back of user's neck, with its top end being substantially aligned with user's vertebrae C3 level, and bottom end being extended to user's vertebrae C7 or lower. Said top end and bottom end are securely attached to the back of user's neck, and maintain a substantially neutral form when user's neck and/or head are in a good upright posture. When user's neck and/or head protrudes forward, said bend sensor may be bent forward, hence to be able to sense user's forward protrusion of neck and/or head, and/or the degree of user's forward protrusion of neck and/or head. When user's neck and/or head protrude forward beyond a predefined tolerance, circuit board may trigger sensory alerts and/or instructions from within the apparatus, and/or one or more of external electronic device, in a similar fashion as discussed above, which can be easily implemented by persons skilled in the art at the time of present invention.

FIG. 19 is a front view of another embodiment of FIG. 18. Vertically erect structure 10 can be applied to the front of user's neck under his chin. When user's neck and/or head protrude forward, corresponding sensors may be bent beyond certain predefined tolerance. Logic and operation controller 37 (not shown) may then trigger sensory alerts and/or instructions from within the apparatus, and/or from one or more of external electronic device, in a similar fashion as discussed in earlier descriptions, which can be easily implemented by persons skilled in the art at the time of present invention.

FIG. 20 is a perspective view of another embodiment of the FHP apparatus 1 of the present Invention, where vertically erect structure, posture sensing and data transmitting functions are built in one thin and small rectangular chip 10 that may be equivalent of vertically-erect structure 10 or may be a small unit that is suitable to be associated with the vertically-erect structure 10. Chip 10 has a miniature size suitable to be securely and discretely placed directly on the skin of the back of user's neck, or on or inside the collar of user's shirt. Preferably said chip may have a dimension of 30 mm×30 mm×5 mm. Or it may have around shape with a diameter of 30 mm and thickness of 5 mm. The mounting mechanism is embodied by adhesive, adhesive tapes, clips, fasteners, magnetic couplers, loops and hooks pieces, or by a collar-like structure that is discussed in FIG. 30. The shells of the Chip 10 may be made of rigid, semi-rigid or flexible material to allow proper posture sensing, effective signal and/or data transmission, accurate and adjustable positioning, secure association with and easy removal from user's body, clothing, or the mounting mechanism 20, good user safety, good user comfort, and/or pleasing visual effect. Sensor or sensors 31 are ideally supported by, held by, printed on, or encased in said chip 10. Circuit board 33, buzzer/vibrator 34, communication module 35, power source or battery unit 36, logic and operation controller 37, and power management integrated circuit 38 and any other necessary electronic components of FHP apparatus 1 are associated with, ideally held by and encased in, said chip 10.

FIG. 21 is a perspective view of another embodiment of the FHP apparatus 1 of the present Invention, wherein the embodiment of FIG. 20 is extended horizontally to form extensions 46 to allow more space for power source 36. Said horizontal extensions 46 may be made to one side or both sides of chip 10. Said extension 46 may be made of flexible or semi-flexible materials such as, but not limited to, plastics, rubber or stainless steel, to house power supply 36 or other component if necessary. Such elongated chip 10 may be capable of securely and discretely be placed on or inside the collar of user's shirt. Preferably it has an outer dimension of no more than 150 mm×30 mm×5 mm. The middle section of 30 mm×30 mm×5 mm may house most of the necessary components of said chip 10, while the extensions 46 to the left and right of the middle section would be mainly devoted to house the necessary power source.

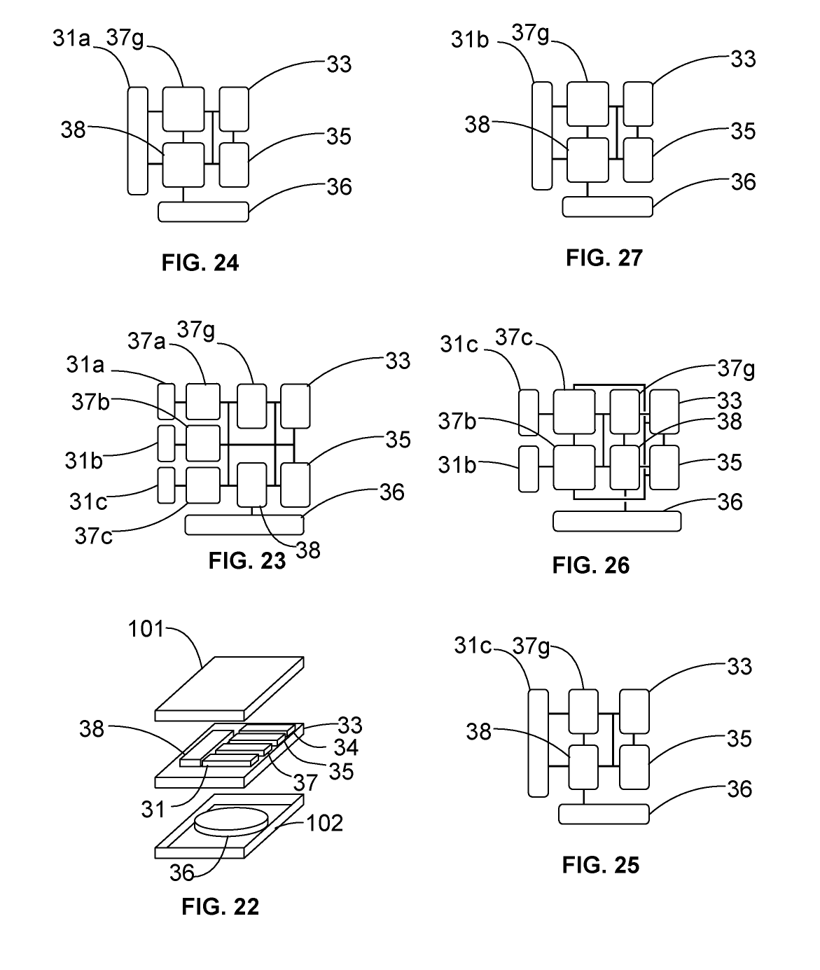

FIG. 22 is a perspective assembly view of said embodiment of the FHP apparatus 1 of the present invention in FIG. 20. Chip 10 comprises functional components such as, but not limited to, upper case shell 101, lower case shell 102, any combination of internal electronic features and/or components detailed in FIG. 23 through FIG. 27, such as sensors 31, including the corresponding sensor processors, circuit board 33, alert unit (buzzer/vibrator) 34, communication module 35, power source 36, general logic and operation controller 37, and power management integrated circuit (PMIC) 38. Said chip 10 is to be applied behind user's neck in such manners as, but not limited to, attached to user's skin through adhesive, or attached on or inside user's collar, or a mounting mechanism.

FIG. 23 is a version of schematic block diagram view of the embodiments of the FHP apparatus of the present invention in FIG. 1 or FIG. 22. Said version of schematic block diagram comprises functional components such as, but not limited to, sensors 31 (including corresponding sensor processors), communication module 35, circuit board 33, general logic and operation controller 37g, power management integrated circuit (PMIC) 38, and power source or battery unit 36. Sensors 31 preferably comprise one or more proximity sensors 31a, one or more accelerometers 31b, one or more angular velocity sensors 31c, or any combination thereof. Said proximity sensors 31a are to sense or detect the position of user's neck and/or head, and user's head and/or neck posture, by substantially continuously monitor the distance between the sensors and the back of user's neck. Said accelerometers 31b and/or angular velocity sensors or gyroscopes 31c are to sense user's state of being, such as, but not limited to, sitting or standing, walking, running, driving, or flying, by monitoring the accelerations and/or angular velocity of user's upper body in one or three axes.

Corresponding sensor processors 37a, 37b, and 37c) are designated to their corresponding sensors 31a, 31b and 31c, suitable for controlling, regulating and organizing the operation of said sensors, such as, but not limited to, transmission and reception of sensing signals, data processing and conversion, calculation of distance, acceleration, tilt, or rotation information according to predetermined logic, algorithm or library, from raw measurement data such as voltage, current, magnetic and/or time-of-flight signals, transmission of the raw or processed data to general logic and operation controller microprocessor 37g, or directly to external device through communication module 35, communication with external devices, as previously discussed.

General logic and operation controller 37g focuses on controlling, regulating and organizing the overall operation of the FHP apparatus 1 that are not controlled, regulated or organized by the sensor processor 37a, 37b, and 37c, such as, but not limited to, data aggregation, processing and conversion, transmission of the raw data, or processed data to external devices through communication module 35, communication with external devices, triggering alarms, etc. as previously discussed.

Details on sensors 31a, 31b, and 31c, including the corresponding sensor processors 37a, 37b, and 37c, circuit board 33, alert unit (buzzer/vibrator) 34, communication module 35, power source 36, general logic and operation controller 37, and power management integrated circuit (PMIC) 38, are similar with the details of the embodiment in FIG. 22. Any component or components such as, but not limited to, sensors 31, communication module 35, circuit board 33, general logic and operation controller 37g, Power management integrated circuit (PMIC) 38, power source or battery unit 36, or any combination thereof, may or may not be incased in case shells 101 and 102, or may be housed in separate case/cases, or may be available to be applied without any casing, whereas said components may be electronically connected or associated or linked, with or without wire.

The calculation of distance information used in determining whether a user's neck has moved is according to predetermined logic, algorithm and/or library, from raw measurement data such as voltage, current, capacitance, magnetic and/or time-of-flight signals, with proximity sensors. The calculation of tilt, inclination, acceleration in one or more axis, used in determining forward tilt of user's head, and/or user's state-of-being information is according to predetermined logic, algorithm and/or library, from raw measurement data such as voltage, current, magnetic, gravity victor and/or accelerations in one or more axis, with accelerometers. The calculation of tilt, inclination, and/or rotation angle information used in determining forward tilt of user's head, user's state-of-being information is according to predetermined logic, algorithm and/or library, from raw measurement data such as voltage, current, magnetic, and/or angular velocity signals in one or more axis, with angular velocity or gyro sensors, Such calculations are simple and obvious to people skilled in the art.

In an example differentiating the state of sitting at home vs the state of sitting in a car may be among the more difficult tasks, but can be relatively easily achieved by monitoring the presence or lack of presence of the strong accelerations along the sagittal axis and periodical accelerations in the transverse axis due to imperfect conditions on the road. And for example, differentiating the state of standing and sitting may be easily determined by monitoring, recording and remembering transitions between the two states due to the accelerations on the vertical axis, and assuming the user remains in the same position before the next transition. In the same principle, the differentiation between sitting at home and sitting in an airplane can also be easily achieved by monitoring, recording and remembering transitions between the two states due to the strong and prolonged acceleration on the axis parallel to airplane's longitudinal axis during departure, and assuming the user remains in the same position before the next transition. Alternatively, the differentiation between sitting at home of office and sitting in a car or an airplane can be accomplished with the help of a GPS sensor that measures the speed of motion of a user. Said chip 10 is to be applied behind user's neck in such manners as, but not limited to, attached to user's skin through adhesive, or attached on or inside user's collar or a mounting mechanism.

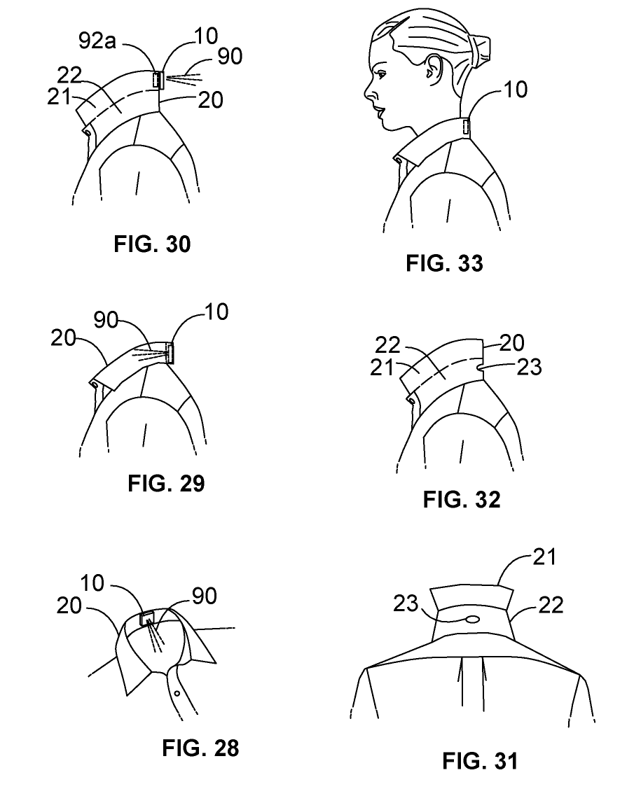

FIG. 24 is another version of schematic block diagram view in FIG. 23, void of sensors 31b and 31c, and their corresponding sensor processors 37b and 37c. Proximity sensors 31a are to monitor and/or measure the forward movement of user's head and/or neck in the sagittal plane. The proximity sensors are selected from the group consisting of capacitive, laser, infrared, inductive, magnetic, sonar, and radar sensors. This embodiment of the present invention is to be mounted behind user's neck, through such means as, but not limited to, attaching to user's collar, or mounted on a mounting mechanism, or as illustrated in FIG. 1, FIG. 10, FIG. 15, FIG. 28 through FIG. 36, and/or FIG. 41 and FIG. 42.

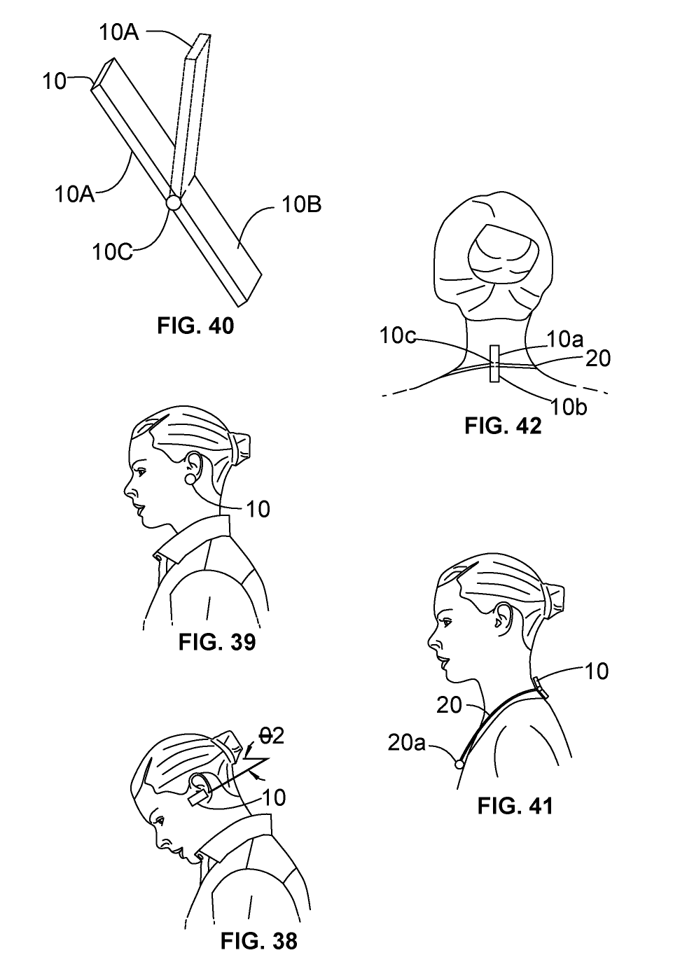

FIG. 25 is another version of schematic block diagram view in FIG. 23, void of sensors 31a and 31b, and their corresponding sensor processors 37a and 37b. Angular velocity sensors 31c are to sense and/or detect user's head and/or neck posture, by substantially continuously monitor the forward tilting angle of user's head. This embodiment of the present invention is to be mounted on one or both sides of user's head, through such means as, but not limited to, ear bud, ear plug, ear loop, ear ring, or as illustrated in FIG. 38, or FIG. 39.

FIG. 26 is another version of schematic block diagram view in FIG. 23, void of sensors 31a, and their corresponding sensor processors 37a. Said angular velocity sensors 31c are to sense and/or detect user's head and/or neck posture, by substantially continuously monitor the forward tilting angle of user's head. Said accelerometers 31b are, may or may not be in combination with said angular velocity sensors 31c, to sense user's state of being, as previously discussed. Combined, sensors 31b and 31c may provide more reliable results in measuring the tilt of user's head and monitoring the user's head posture. This embodiment of the present invention is to be mounted in or on or around user's ear(s) on one or both sides of user's head, through such means as, but not limited to, ear bud, ear plug, ear loop, ear ring, or as illustrated in FIG. 38, or FIG. 39.

FIG. 27 is another version of schematic block diagram view in FIG. 23, void of sensors 31a and 31c, and their corresponding sensor processors 37a and 37c. Said accelerometers 31b are, to sense the tilts of user's head, user's state of being, as previously discussed. This embodiment of the present invention is to be attached to user's ear(s) on one or both sides of user's head, through such means as, but not limited to, ear bud, ear plug, ear loop, ear ring, or as illustrated in FIG. 38, or FIG. 39.

FIG. 28 is a front view of an embodiment of the FHP apparatus 1 of the present invention in FIG. 20 or FIG. 21, mounted on a collar. Compared with the embodiment in FIG. 1, the chip 10 is the equivalent of the vertically erect structure 10, and the collar 20 is the equivalent of the mounting mechanism 20. Collar 20 may be the integral collar of a normal shirt, or a conventional stand-alone and/or removable collar that can be worn on user's body independent of what user may already be wearing. Chip 10 is securely affixed to the symmetrical middle of collar 20, with its sensor and/or sensors 31 substantially aligned with, and/or arranged close to the top edge of collar 20, corresponding to vertebrae C3 or the hair line at the back of user's head when in use.

In one embodiment, sensors may be outside of the casing of chip 10 and may be directly associated with collar 20 and substantially aligned with, and/or arranged close to the top edge of the symmetrical middle portion of collar 20, through such methods as, but not limited to, being printed on, being glued on, being pressed on, being embossed, being woven in, being clipped on, being fastened on, or being attached or secured on using magnet/magnets. In such embodiment, sensors may be associated with collar 20 in such ways as, but not limited to, on the inner surface of inner wall (portion of collar below folding line) of collar 20, on the outer surface of inner wall of collar 20, in the inner wall of collar 20, or on the inner surface of outer wall (portion of collar above folding line) of collar 20. The sensing surface 90 of chip 10 faces toward the front of the shirt, i.e. user's neck when in use. Suitable sensors for this embodiment are as those previously discussed in FIG. 23 or FIG. 24.

FIG. 29 is a side view of an embodiment of the FHP apparatus 1 of the present invention in FIG. 20 and/or FIG. 21, mounted inside the fold of a collar. Compared with FIG. 28, chip 10 is mounted inside the fold of a collar instead of on the surface of a collar. The sensing surface 90 of chip 10 faces toward front of the shirt, i.e. user's neck. Chip 10 is securely affixed to the symmetrical middle of collar 20, with its sensor and/or sensors substantially aligned with, and/or arranged close to the top edge of collar 20, corresponding to the hairline at the back of user's head when in application. Suitable sensors for this embodiment are as those previously discussed in FIG. 23 or FIG. 24.

FIG. 30 is a side view of an embodiment of the FHP apparatus 1 of the present invention in FIG. 29, mounted on the back of the upper portion 21 of collar that is then to be folded down to be secured inside the fold of the collar 20. Before the upper portion 21 of the collar 20 being folded down to cover lower portion 22 of collar 20 and to secure chip 10, sensing surface 90 of chip 10 faces towards the back of the shirt, to allow the sensing surface 90 to face towards the front of the shirt, i.e. user's neck after the upper portion of the collar is folded down backwards as per people's common practice in daily lives. Suitable sensors for this embodiment are as those previously discussed in FIG. 23 or FIG. 4.

Chip 10 may be secured to the collar by such means as, but not limited to, a sufficiently strong magnet on the opposite side of collar, a fastener to be clipped to the edge of the collar, or a pin to punch through the collar and secured by a lock.

FIG. 31 is a rear view of a shirt collar with a special hole 23 preferably opened in the upper center part of the lower portion 22 of the collar 20 to improve the reliability and/or accuracy of distance sensing between the proximity sensor and the back of user's neck. Said hole may be void of any fabric or material, or be equipped with a layer of fabric with predetermined material, thickness, color, texture, etc. to assure reliable penetration of sensing signals and accurate distance sensing. The diameter of said hole may be 10-15 mm to sufficiently expose the proximity sensors to their target—back of user's neck.

FIG. 32 is a side view of a shirt collar 20 with a special hole 23 as per FIG. 31. Said shirt collar may be a specially made shirt collar for the sole purpose of holding chip on or inside itself to monitor the sagittal motion of user's head and neck. Said shirt collar may be void of the unnecessary parts for said purpose such as, but not limited to, sleeves, lower portions of the front and rear panels of the shirt.

FIG. 33 is a side view of an embodiment of the FHP apparatus 1 of the present invention in FIG. 20, FIG. 28, FIG. 29, FIG. 30, or FIG. 31, mounted on a collar and applied to a user. In its normal condition when being used by a user, such collar 20 generally stands sufficiently upright and assumes natural state of collar that aligns with natural curvature of user neck when user's head is in a good and upright position, with its highest tip properly aligned with, stays close to the hairline at the back of user's head, and substantially mimic the sagittal disposition or standing of user's neck when in a upright head/neck posture, and substantially maintains such a position throughout the day, as long as the user is in a upright posture whether sitting, standing, and/or walking. Said highest tip is normally the top edge in the middle section of the collar 20 that is next to the back of user's neck, when worn by the user in a desirable neck/head posture.

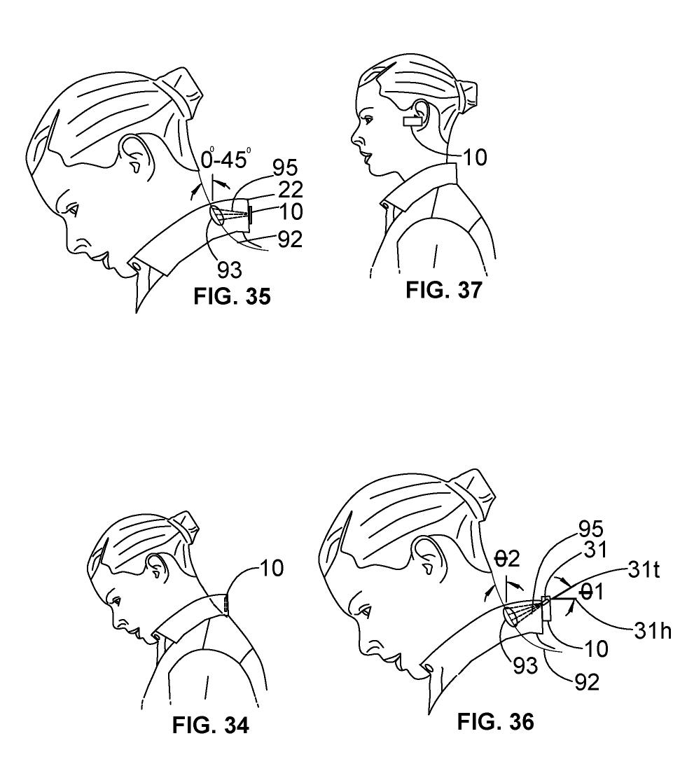

FIG. 34 is a side view of an embodiment of the apparatus of the present invention in FIG. 33, when user's head protrudes forward. A gap emerges between the back of user's neck and upper portion of user's collar 20 where sensors 31 are located, when user's head moves or tilts forward. Sensors and sensor processors measure such widening gap and inform the general logic and operation controller 37g of such gap. Said general logic and operation controller 37g shall control, regulate and coordinate the function of the FHP apparatus in the manner as previously discussed. Measurement and/or transmission of data and/or information relating to user's head and/or neck position is preferably at a predetermined rate such as, but not limited to, twice per second. Said rate may vary according to the user's state of being. For example, the rate may be once every 3 seconds when user is sitting at home or in an office, 3 times per second when user is walking, and 5 times per second when user is running. The extend of such gap, and/or the period in which such gap occur are measured, monitored and analyzed by chip 10 and/or external devices, such as but not limited to smartphone, smart wrist band, smart finger ring, or cloud server. The decision of whether, when, where or how to trigger sensory alert from the chip 10 and/or from any external device may be made by an app on an external device, or by the general logic and operation controller of chip 10, according to predetermined logic and/or algorithm. Said sensory alert may be delivered by chip 10, or one or more said external devices. Said sensory alert may be delivered with some delay, such as, but not limited to, 10 seconds or 3 minutes, to tolerate user's need to knowingly and temporarily protrude and/or tilt their head forward, and to avoid over-alert. Said delay may be adjusted during use or preset ahead of use by user, according to their habit, comfort, desired tolerance of postural deviation, and/or professional and/or personal need. Such presetting and/or adjusting function could be enabled via said external device and/or chip 10. In case of group users, such presetting and/or adjusting could be centrally and uniformly controlled by external device or devices, instead of by individual user. Hence, chip 10, collar 20, external devices, and their analyzing, controlling and operating software may form an integrated ecosystem for helping user and users improve their head and/or neck posture. Such an ecosystem may enable user or users easily and effectively monitor and/or improve their head and/or neck posture.

An appropriate method of applying said system is critical. Such appropriate method of applying said system may include, but not limited to, such steps as

1. associating chip 10 on collar 20 and arranging the sensor or sensors be substantially aligned with and placed close to the top edge of middle section of said collar as shown in FIG. 28, FIG. 29, or FIG. 30,
2. applying collar 20 to user's body as a fitting collar and/or a fitting shirt that secures a reliable and snug fit, button up all necessary buttons,
3. assuring user to pose and maintain in a good and upright head and/or neck posture desired by the user or advised by a professional, until the following set up steps are successfully completed,
4. assuring chip 10 is substantially aligned with center line of user's cervical spine, and assuring chip 10 is in touch with and/or substantial close to the back of user's neck,
5. activating and/or calibrating chip 10 and necessary external device and/or devices for reference and/or targeted upright head and neck posture, if necessary,
6. accepting the reference, if necessary,
7. setting desired tolerance of spatial deviation of user's head and/or neck from the reference and/or targeted head and/or neck posture, if necessary,
8. setting desired time delay of sensory alert when head and/or neck posture deviates from reference, if necessary,
9. proceeding to normal daily activities,
10. when sensory alert is triggered, reverting head and/or neck protrusion and/or tilt. Sensory alert should stop when user's head and/or neck is identical or substantially close to the pre-set reference posture. and
11. repeating any or all above step or steps anytime user desires to monitor and/or improve their head and/or neck posture.

FIG. 35 is a side view of a simplified illustrative simulation of proximity sensing arrangement of an embodiment of the FHP apparatus of the present invention according to FIG. 20, FIG. 28, FIG. 29, FIG. 30, FIG. 31, or FIG. 33. Chip 10 is to measure and monitor the distance between the chip and the target spot 93 on back of user's neck 92 in sagittal plane using proximity sensors. Chip 10 is substantially vertical, with its main viewing/sensing beam 95 substantially horizontal. Said target surface spot 93 is substantially located on the gravity line of user's neck and at the same horizontal level as the sensors 31 inside the chip 10 when user's neck tilts forward, said target surface spot 93 may slide upward on the back of user's neck since the position of sensors remain unchanged. Between sensors 31 and back of user's neck there may exists a layer of fabric that may be a lower portion 22 of user's collar or a fabric of predetermined materials, thickness, opacity, color, etc. Target surface spot 93 is bare skin of user's neck, has a spot size of 10-20 mm in diameter, and may tilt away in a range of 0-45 degrees. The distance between the sensors 31 and the target surface spot 93 may vary in a range of 0-60 mm. Sensors 31 are preferably capable of sensing said distance with an accuracy of +/−2 mm in the given arrangement. The total external size of chip is preferably no more than 30 mm×30 mm×5 mm, or 150 mm×30 mm×5 mm. To sufficiently penetrate said layer of fabric 22, sensors may preferably be based on such technologies as, but not limited to, infrared sensing, or capacitive sensing, or any combination thereof. To substantially focus on the small target spot, infrared sensors may be equipped with collimating lens/lenses, and capacitive sensors may be equipped with guard rings.

FIG. 36 is a side view of a simplified illustrative simulation of proximity sensing arrangement of an embodiment of the FHP apparatus of the present invention according to FIG. 20, FIG. 29, FIG. 30, FIG. 31, FIG. 33, and FIG. 35. For optimum reliability and accuracy of proximity sensing, especially in case of optical distance sensors such as infrared or laser sensors, the target surface spot 93 is preferably perpendicular to the main viewing/sensing beam 95 of sensors 31 to allow maximum signal feedback to sensors 31. To facilitate said maximum signal feedback, sensors 31 may be tilted downward relative to the body of chip 10 from a horizontal position 31H to a tilted position 31T with a tilt angle 81 of sensors roughly equaling to the medium tilt angle 82 at which user's neck tilts forward.

FIG. 37 is a side view of an embodiment of the apparatus of the present invention according to FIG. 20, and FIG. 25 or FIG. 26 or FIG. 27. Chip 10 is attached to user's ear like an ear phone or an ear plug. Its accelerometers and/or angular velocity sensors or gyroscope may sense and measure the tilt or tilting motions of user's head in sagittal plane when user's head tilts forward. Said tilt and/or tilting motion is to be measured and/or detected by said accelerometers and/or angular velocity sensors. When the predetermined spatial and/or time tolerances for the forward tilting motion of user's head have been exceeded, chip 10 may trigger sensory alert to user directly or through one or more associated external devices such as, but not limited to, smartphone, smart wristband, radio in a car or airplane, or black out the screen of user's smartphone and/or computer.

FIG. 38 is a side view of an embodiment of the apparatus of the present invention similar to FIG. 37, wherein user tilts his head forward. When user's head tilts forward, chip 10 rotates forwards, and angle θ2 emerges. Accelerometer, and/or angular velocity sensor tilts or rotates forward accordingly. As a result, said accelerometer and/or angular velocity sensor sense and measure the tilt and/or the tilting motions θ2 of user's head in sagittal plane, hence monitor user's head posture. When said accelerometer and/or angular velocity sensor or gyroscope detect the forward tilts θ2 of user's head exceeding predetermined spatial and/or time tolerances, chip 10 may trigger sensory alert to user directly or through one or more associated external devices such as, but not limited to, smartphone, smart wristband, radio in a car or airplane, or black out the screen of user's smartphone and/or computer.

FIG. 39 is a side view of another embodiment of the apparatus of the present invention according to FIG. 37 wherein chip 10 is attached to user's ear like a decorative ear ring. The function of sensing and/or measuring the forward tilt or tilting motion of user's head and user's head posture is similar to the way described in FIG. 37 and FIG. 38.

FIG. 40 is a perspective view of another embodiment of the apparatus of the present invention. Vertically erect structure 10 comprises an upper section 10a, and lower section 10b and a pivot section 10c. Pivot section 10c allows easy adjustment of upper section 10a so that the upper end of 10a can be adjusted to be substantially in slight touch with or in substantial close proximity to the back of user's neck, when user is in a good upright posture with his neck and head. Pivot section 10c is capable of holding the position or angle of upper section 10a securely without undesired pivot movement either forward or backward while in operation. Pivot section 10c is also capable of holding a chain that loops around user's neck like a necklace. Pivot section 10c is substantially aligned with the base of user's neck, i.e. vertebrae C7/T1. Sensors 31 may be integrated with the upper section 10a and substantially aligned with the top edge of said upper section 10a. Buzzer/vibrator 34 and power source or battery unit 36 may be integrated with lower section 10b. Communication module 35, circuit board 33, general logic and operation controller 37 or 37g, and/or power management integrated circuit 38 may be integrated with either upper section 10a or lower section 10b depending on special or other considerations.

FIG. 41 is a side view of said embodiment of the apparatus of the present invention in FIG. 40, applied on a user. When a collar 20 of FIG. 28 through FIG. 33 is not available or not desirable by certain user, this embodiment may enable said user easily wear the FHP apparatus 1 to monitor and improve head and/or neck posture. Necklace-like chain 20 functions as mounting mechanism to secure and align device 10 on user's neck. Upper section 10a concaves towards or with user's lordosis, and may be substantially in touch with or close to the back of user's mid to upper neck when user is in a good and upright head/neck posture. Pivot section 10c may be substantially aligned with the base of user's neck around vertebrae C7/T1. Sensor and/or sensors may be associated with the upper section 10a and aligned with top edge of said upper section 10a. To improve the security of holding device 10 in proper position, said chain 20 may be equipped with a suitable balance weight 20a in its front portion, to counter balance the weight of device 10 at the back of user's neck.

FIG. 42 is a rear view of said embodiment of the apparatus of the present invention in FIG. 40, applied on a user. Vertically erect structure 10 is placed behind user's neck, and aligned with the center of the back of user's neck.

While preferred embodiments of a method and apparatus for monitoring neck posture and gait have been described and illustrated in the detail, it is to be understood that numerous modifications can be made to the embodiments of the present invention without departing from the spirit thereof.

What is claimed is:

1. An apparatus for real time monitoring of head posture, comprising:
at least one processor;
at least one sensor configured to sense the position and posture of a wearer of the apparatus;
wherein the at least one processor receives and processes input from the at least one sensor;
wherein the apparatus is worn above the shoulders of the wearer.

2. The apparatus of claim 1, further comprising an alarm module.

3. The apparatus of claim 1, further comprising:
a wireless transmission module in communication with the at least one processor and configured to communicate with one or more external device.

4. A posture monitoring ecosystem for monitoring, communicating, recording, analyzing, tracking, and reviewing the posture of a user, comprising:
at least one sensor configured to sense the position and posture of a wearer of the device;
at least one processor, wherein the at least one processor receives and processes input from the at least one sensor;
one or more external electronic devices, whereby such external electronic devices communicate directly or indirectly with the apparatus through the wireless transmission module; and
at least one app resides on the one or more external electronic devices, wherein the at least one app controls the related operations of the one or more external electronic devices.

5. The posture monitoring ecosystem of claim 4, wherein one or more external electronic devices may trigger alarm and indicate the wearer's posture needs attention, or become unusable, until the wearer's posture is corrected.

6. The apparatus of claim 2, wherein the alarm module comprises at least one alarm mechanism selected from the group consisting of auditory, vibratory, and visual alarm mechanisms.

7. The apparatus of claim 1, wherein the at least one processor processes posture information received from the at least one sensor and compares the received posture information with predetermined spatial and time tolerances, and when the tolerances are exceeded, the processor controls an alarm module to indicate to the wearer and/or another party that attention to neck posture is needed.

8. The apparatus of claim 1, wherein the at least one sensor comprises selections from the group consisting of proximity sensors, touch sensors, accelerometers, and angular velocity sensors or gyroscopes.

9. The apparatus of claim 8, wherein the at least one sensor measures angles and distances in the sagittal plane.

10. The apparatus of claim 1, further comprising a mounting mechanism.

11. The apparatus of claim 10, wherein the mountain mechanism comprises the selections from the group consisting of loop structure, shirt collar, neck band, neck chain, strap, magnetic clip, adhesive tape, ear plug, ear ring, neck tie, and scarf.

12. The apparatus of claim 10, further comprising a collar attached to the mounting system, wherein the collar retains the at least processor, at least one sensor, and wireless transmission module.

13. The apparatus of claim 10, wherein the mounting mechanism comprises a vertically erect structure to which the at least one sensor is attached.

14. The apparatus of claim 13, wherein the mounting mechanism includes a hinge that operates to maintain a sufficient proximity between the upper portion of the vertically erect structure and the back of the wearer's neck.

15. The apparatus of claim 10, wherein the mounting mechanism comprises front pieces that extend toward the wearer's front, up to wearer's second rib.

16. The apparatus of claim 1, further comprising a chip attached to the at least one sensor, the chip configured to attach to a shirt collar worn by the wearer.

17. The apparatus of claim 1, further comprising a chip attached to the at least one sensor, the chip configured to attach to the wearer's ear.

18. The apparatus of claim 1, further comprising a chip aligned with the at least one sensor, wherein the at least one sensor is configured to measure and monitor the distance between the chip and a target spot on the back of the wearer's neck using one or more proximity sensors, wherein the chip is substantially vertical, and has a viewing/sensing beam that is substantially horizontal, wherein the target surface spot is located substantially on a gravity line of the wearer's neck and at the horizontal level of the at least one sensors aligned with the chip when wearer's neck tilts forward.

19. A method of correcting a person's neck posture, comprising:
a) providing at least one sensor;
b) monitoring a person's neck posture by the at least one sensor worn by a person, by at least one of:
1) monitoring the distance in sagittal plane; and
2) monitoring the angle in sagittal plane;
c) determining that the person's neck posture needs attention; and
d) performing at least one of:
1) drawing attention to the person's neck posture; and
2) compromising an external device's function,
until the person's neck posture is corrected.

20. The method of claim 20 further comprises a step of communicating with one or more external electronic devices.
